(19) United States
(12) Patent Application Publication
(10) Pub. No.: US 2011/0117266 A1
(43) Pub. Date: May 19, 2011

(54) Medical Device Coating System

(75) Inventors: Thomas Mark Marron, Apple Valley, MN (US); Michael Sean Owens, Richfield, MN (US); Mark Steven Smith, Coon Rapids, MN (US); James Edward Lasch, Oakdale, MN (US); Amod Shridhar Modak, Plymouth, MN (US); Andy Dubel, Rogers, MN (US)

(73) Assignee: Boston Scientific Scimed, Inc., Maple Grove, MN (US)

(21) Appl. No.: 12/839,949

(22) Filed: Jul. 20, 2010

Related U.S. Application Data
(60) Provisional application No. 61/226,782, filed on Jul. 20, 2009.
(60) Provisional application No. 61/247,829, filed on Oct. 1, 2009.

(51) Int. Cl. A61L 33/00; B05D 1/28; B05D 7/00; B05C 1/02

(52) U.S. Cl. 427/2.1; 118/232; 118/209

(57) ABSTRACT

A system for coating a medical device comprises a transfer web, a metering web. The webs are each advanced in a downstream direction toward a gap defined by the advancing webs. A coating solution applicator is configured to apply a coating solution at a staging area at a position upstream of the gap. A medical device retaining mechanism is positioned at a coating application area of the transfer web, at a position downstream from the gap.

### CROSS-REFERENCE TO RELATED APPLICATIONS

This application claims the benefit of U.S. Provisional Patent Application No. 61/226,782, filed Jul. 20, 2009, and this application claims the benefit of U.S. Provisional Patent Application No. 61/247,829, filed Oct. 1, 2009, the entire contents of which are hereby incorporated herein by reference.

### STATEMENT REGARDING FEDERALLY SPONSORED RESEARCH

Not Applicable

### BACKGROUND OF THE INVENTION

1. Field of the Invention

In some embodiments this invention relates to a system, including apparatuses and their methods of use, for coating a medical device and to the medical devices produced by the aforementioned system.

2. Description of the Related Art

One or more surfaces of a medical device may be coated with one or more of a variety of therapeutic agents in order to provide for the localized delivery of the agent(s) to a targeted location within the body, such as an artery or other body lumen. Such localized drug delivery may be achieved, for example, by coating balloon catheters, stents or other implantable prostheses with the therapeutic agent(s) to be locally delivered.

The coating(s) on medical devices may provide for controlled release of a drug, and/or provide other benefits such as improved radiopacity, lubriciousness, biocompatibility, etc.

Often the therapeutic coating to be applied to the medical device comprises a polymeric agent which contains a dissolved and/or suspended bioactive agent or drug. The polymer/drug aspect of the coating is itself often dissolved in a solvent solution. This mixture is applied to the medical device through a variety of mechanisms such as by spray coating (an example of which is described in U.S. Pat. No. 6,669,980), droplet deposition (examples of which are described in U.S. Pat. No. 7,048,962, U.S. Publication 2006/0172060 and U.S. Publication 2006/0217801), roll coating (examples of which are described in U.S. Pat. No. 6,984,411 and U.S. Pat. No. 7,344,599), emersion or dip coating (an example of which is described in U.S. Pat. No. 6,919,100), etc. The entire content of each of the aforementioned patents and publication is incorporated herein by reference.

Following the application of the solvent/polymer/drug mixture, the solvent evaporates to leave a dry coating of the polymer/drug agent on the treated surface(s) of the medical device.

Drawbacks however, exist in many of the known systems for applying a coating to a medical device. These drawbacks include the capacity to coat specific surface of the medical device (e.g. providing only the abluminal surface of a stent with a coating while avoiding coating the luminal surface; the inability to accurately apply the coating to the medical device uniformly, the inability for specific methods and systems to efficiently and repeatedly coat multiple medical devices with consistent quality, a high degree of downtime and lengthy change over times between coating cycles, etc.

There is therefore a need for alternative coating methods for medical devices.

The art referred to and/or described above is not intended to constitute an admission that any patent, publication or other information referred to herein is “prior art” with respect to this invention.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a system and method for coating a medical device such as a catheter, balloon or implantable prosthesis such as a stent.

In at least one embodiment the system incorporates one or more “web” pathways upon which the coating solution is applied. The webs advance along pathways which converge at a gap, through which the coating solution is passed in order to regulate characteristics of the coating, particularly its thickness. The regulated (metered) coating solution is then advanced to a region where a medical device is passed through a portion of the coating solution thereby providing the medical device with a coating having a substantially uniform thickness.

In some embodiments, the height of the gap controls the thickness of the coating solution to be applied to the stent. The height of the gap is adjustable to provide a variety of coating thicknesses. In addition, the speed of the web can provide additional control of coating thickness.

In some embodiments the web material acts as barrier to prevent the coating from contacting any components of the coating system, and only coming in contact with the medical device to be coated.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a frontal view of an embodiment of the present invention.

FIG. 1a is a frontal view of an embodiment of the present invention.

FIG. 2 illustrates the direction of the transfer web pathway as shown in the embodiment of FIG. 1.

FIG. 3 is a sectional view of the embodiment shown in FIG. 1 illustrating components comprising the metering web pathway, and the pathway's direction.

FIG. 4 is a frontal view of an alternative embodiment of the system shown in FIG. 1.

FIG. 5 shows an embodiment of the transfer web pathway including a drive mechanism/spindle.

FIG. 6 is a detailed depiction of the gap defined by the metering web (via the blade) and the transfer web (via the base) shown in FIGS. 1-4 through which a quantity of coating solution is passed before application onto a medical device.

FIG. 7 is a close-up perspective view of the coating deposition system shown in FIGS. 1-4.

FIGS. 8 a-c are a series of top-down views of the transfer pathway depicting the deposition of a quantity of coating solution at a staging area of the transfer web shown in FIGS. 1-4.

FIG. 9. is a top-down view of the transfer pathway showing the coating patch after it has passed through the metering gap shown in FIGS. 1-4 and 6.

FIGS. 10a-10b are side views of a medical device being rolled through a working portion of the coating patch depicted in FIG. 9.

FIG. 11 is a top-down view of the transfer pathway showing the coating patch following the application of coating to the stent, such as depicted in FIG. 10.

FIG. 12 is a longitudinal cross-section of a non-uniform metering gap caused by blade misalignment relative to a base.

FIG. 13 is a longitudinal cross-section of a non-uniform metering gap caused by non-uniformity in a base.

FIG. 14 is a front view of a non-uniform metering gap detected using lighting from a backside of a blade.

FIG. 15 is conceptual diagram illustrating relative positions of an operator, a metering gap, and a light source for detecting non-uniformity in the gap.

FIG. 16 is a graph illustrating a coating solution patch thickness along its length.

FIG. 17 is a graph illustrating a transfer web thickness and a combination of the web thickness and a coating solution patch thickness along its length.

FIGS. 18A and 18B are graphs illustrating that a force applied to a medical device during a coating process varies over time.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated. It should also be understood that components or features of the present invention shown or described in one embodiment can be incorporated into other embodiments as desired.

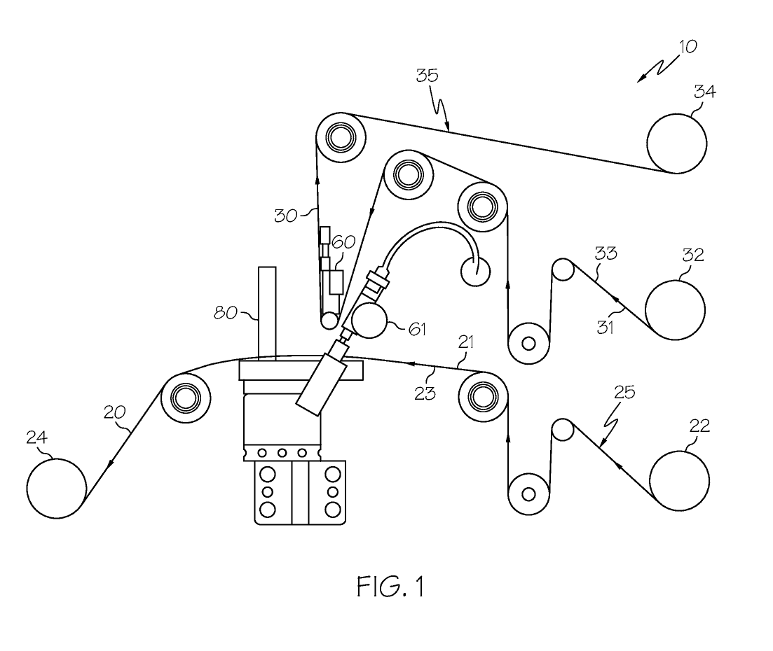

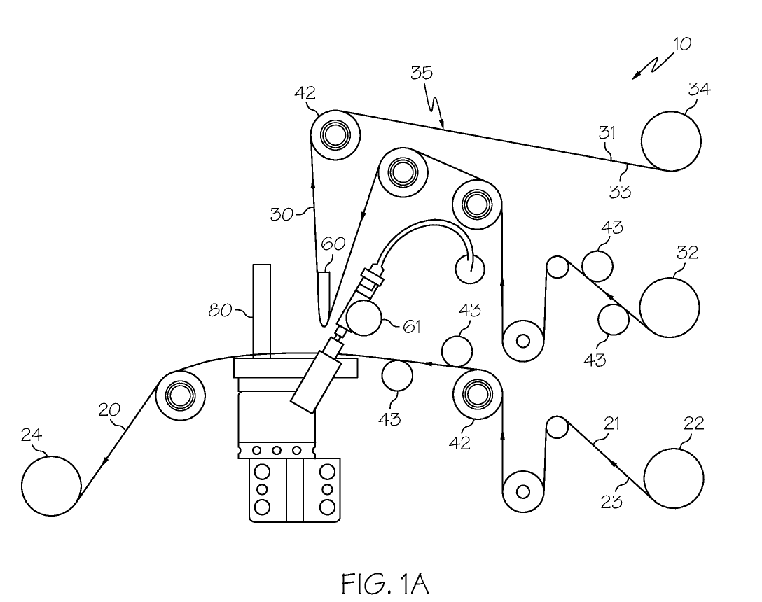

As indicated above, the present invention is embodied, in at least one form, as an apparatus or system for coating a medical device. An example of such a system is shown in FIGS. 1-4. In the embodiment shown, the coating system 10 comprises a transfer web 20 and a metering web 30 which form a metering gap 54 for the metering of a quantity of coating solution passed therethrough.

Transfer web 20 and metering web 30 can be constructed of the same or different materials. In some embodiments the material of the webs 20 and/or 30 is a material resistant to the effects of the solvents and other materials commonly present in coating solutions. The material also resists migrating into the coating solution, so as to ensure the consistent composition of the solution throughout the coating process.

In at least one embodiment, webs 20 and 30 are each composed, at least partially, of biaxially oriented polypropylene (BOPP). Other suitable materials for use in the manufacture of webs 20 and/or 30 include but are not limited to: polyethylene naphthalate (PEN), Polyethylene terephthalate (PET), polypropylene (PP), polyethylene (PE), polytetrafluoroethylene (PTFE), expanded polytetrafluoroethylene (ePTFE) and combinations thereof. In some embodiments webs 20 and/or 30 are comprised of coated films or papers that can include functional coatings for scratch resistance, ease of transport, and/or other desired characteristics.

The thickness of the webs 20 and 30 can be any thickness desired. In some embodiments, the thickness of one or both webs is about 0.1 μm to about 100 μm. In some embodiments the thickness of one or both webs is between about 10 μm and about 50 μm. In at least one embodiment, one or both webs 20 and 30 has a thickness of about 25 μm. In at least one embodiment, one or both webs 20 and 30 has a thickness between about 5 μm to about 10 μm.

The width of each web 20 and 30 can also be provided in any width desired. In some embodiments, the width of one or both of the webs 20 and/or 30 is a function of the length of the medical device 100 to be coated using the system 10. The web width is at least as long as the length of the medical device. In at least one embodiment webs 20 and/or 30 have a width of between about 40 mm and about 60 mm. In at least one embodiment the width of each web 20 and 30 is about 50 mm.

In the system 10 shown, transfer web 20 extends between a source mechanism 22 and a receiving or end mechanism 24. The transfer web 20 extends between mechanisms 22 and 24 along a transfer web pathway 25. Similarly, the metering web 30 extends between a metering web source mechanism 32 and a metering web end mechanism 34 following a metering web pathway 35 as shown.

The source mechanisms 22 and 32 may be any type of device capable of providing a constant output of web 20 or 30 from the respective source mechanism; whereas each end mechanism 24 and 34 is limited only by its capacity to accept the web from the respective source mechanism in a similarly constant manner. For example, in the embodiment shown in FIGS. 1-4 the source mechanisms 22 and 32 are spools or rollers which have a predetermined length of web contained thereon and the end mechanisms 24 and 34 are, at least initially empty spools or rollers which receive the appropriate web 20 or 30.

The source mechanisms 22 and 32, which for the purposes of the depicted embodiment are hereinafter referred to as source rolls or rollers, may contain any quantity (length) of web desired. Considerations that limit the quantity of web material on a roller include: the total weight of the roll and web (a drive mechanism, discussed below, must be capable of rotating the roller), the initial diameter of the web (must be sufficiently small to allow rotation and advancement of the web without interference), etc. These considerations apply equally to the end mechanisms 24 and 34 (hereinafter referred to as end or receiving rolls or rollers) as they will eventually accept and accumulate the web received from appropriate source roll 22 or 32.

As previously mentioned, the rollers, (in the case of the transfer web 20: source roller 22 and end roller 24, and in the case of the metering web 30: source roller 32 and end roller 34) are actuated so as to advance the webs along the respective transfer web pathway 25 and metering web pathway 35. In some embodiments, selective control of the speed of the web advancement may be advantageous. In the embodiment shown in FIGS. 1-4 the webs can be advanced along their pathways at rates from about 1 mm/sec to about 1200 mm/sec. In some embodiments the rates are more typically about 100 mm/sec to about 150 mm/sec. The different pathways 25 and 35 may be advanced at the same or different speeds as desired. In at least one embodiment the metering web 30 can be held stationary during all or some period of the coating process.

The mechanism for rotating the rollers can be any device capable of engaging the rollers or webs and imparting movement thereto (hydraulic pump(s), electric motor(s), etc). Various control mechanisms and sensors can also be employed in the system to provide regulation of the web speed relative to the diameter of the rollers at a given time, web tension, etc.; as well as to start web advancement and stop it as desired during the coating process.

In some embodiments the speed of the transfer and metering webs can be driven independently by servomotors incorporated within or operatively engaged to en rollers 24 and 34 and/or source rollers 22 and 32. The rotary speed of each drive required to achieve the desired linear speed of the webs can be initially determined by measuring the web thickness at 22 and 32 independently, calculating the appropriate circumference for each web, therefore allowing for calculation of the theoretical rotary speeds of the servomotors to achieve the desired linear speeds for both the transfer and metering webs. The measurement of the web thickness can be performed by various means. In at least one embodiment it is accomplished optically, e.g. interferometer. The linear speed of each web can be controlled by in-line linear encoders relaying the measured linear speed information to the servomotors, and adjust the rotary speeds if the linear speed is outside of a defined tolerance for a defined period of time. In some embodiments the tensions of the metering and transfer webs are modulated independently by applying a magnetic break at rollers 22 and 32. The tensions of the transfer and metering webs can be controlled by employing an in-line tension measurement device that measures tension at a given point along the metering and transfer web pathways, and relaying this information to rollers 22 and 32 and/or rollers 24 and 34. If the tension of either web are outside a prescribed tolerance for a given period of time then tension can be modulated by applying or releasing the breaks at 22 and 32.

In at least one embodiment a single drive mechanism for each web pathway 25 and 35 may be utilized externally from the rollers. An example drive mechanism for transfer web pathway 25 is shown in FIG. 5 wherein the transfer web 20 passes from source roller 22 to end roller 24 and around a drive spindle 40. Actuation of the drive spindle 40 by a motor or other mechanism will allow the web 20 to move along the pathway 25 in the direction indicated. It should be noted however, that even with a single drive mechanism, the speed and even the direction, of the web advancement can be reversed if desired. Metering web 30 can be similarly advanced and manipulated.

In some embodiments, adjustment spindles 42, such as are shown in

FIGS. 1-3 are employed on one or both pathways 25 and 35 to direct the pathways as desired and to maintain and regulate the tension of the webs 20 and/or 30.

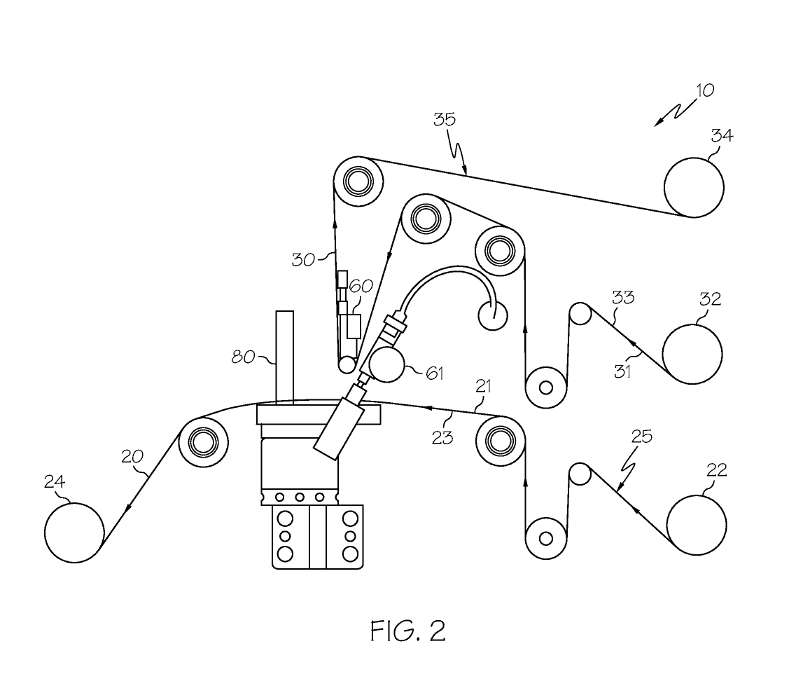

As is shown in FIG. 1a, in some embodiments, one or more of the spindles is configured as a debris removal spindle 43 and is positioned to come into contact with the outside surface (e.g. the working surface, or the surface with which the coating solution comes into contact) and/or the inside surface of one or both webs (transfer web 20 includes outside surface 21 and inside surface 23; metering web 30 includes outside surface 31 and inside surface 33). In at least one embodiment, an example of which is shown in FIG. 2, one or more debris removal spindles 43 is in contact with only the inside surface 23 and/or 23 of one ore both webs 20 and 30.

A debris removal spindle 43 will include a tacky material that comes into contact with the web(s) in order to pick up any small debris or foreign matter such as dust, hair or other particles. In at least one embodiment, the debris removal spindle 43 comprises a surface that includes urethane.

In some embodiments, one or both webs 20 and 30 are subjected to active ionization to reduce or eliminate static electricity. Such active ionization can be achieved through the use of one or more ionizing unit positioned upstream of the staging area 52.

Once the webs 20 and 30 are properly positioned and secured within the system, such as is shown in FIGS. 1-4 the coating process may be initiated.

### Process Summary

Before advancement of the webs, or during a point when at least the transfer web 20 is not moving (stopped) by a control mechanism (not shown), an initial quantity of coating solution 50 is deposited from an applicator onto an outside surface 21 of the transfer web 20 at a staging area 52. In some embodiments the applicator 61 is a syringe. Once the initial quantity of coating solution 50 is applied, the transfer web 20 is advanced in order to move the solution 50 from the staging area to a metering gap 54. The initial quantity of coating solution 50 is then passed through the metering gap 54.

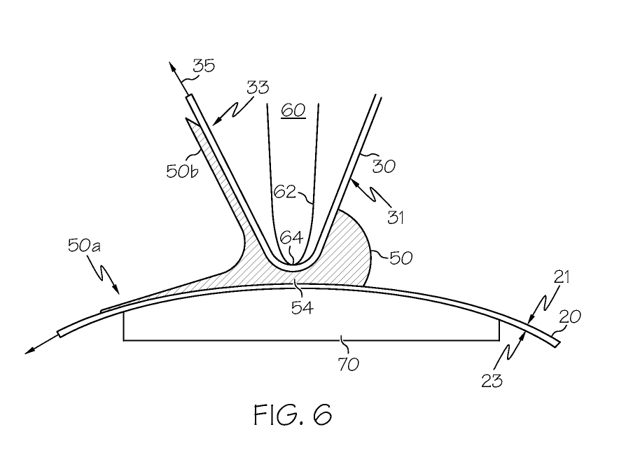

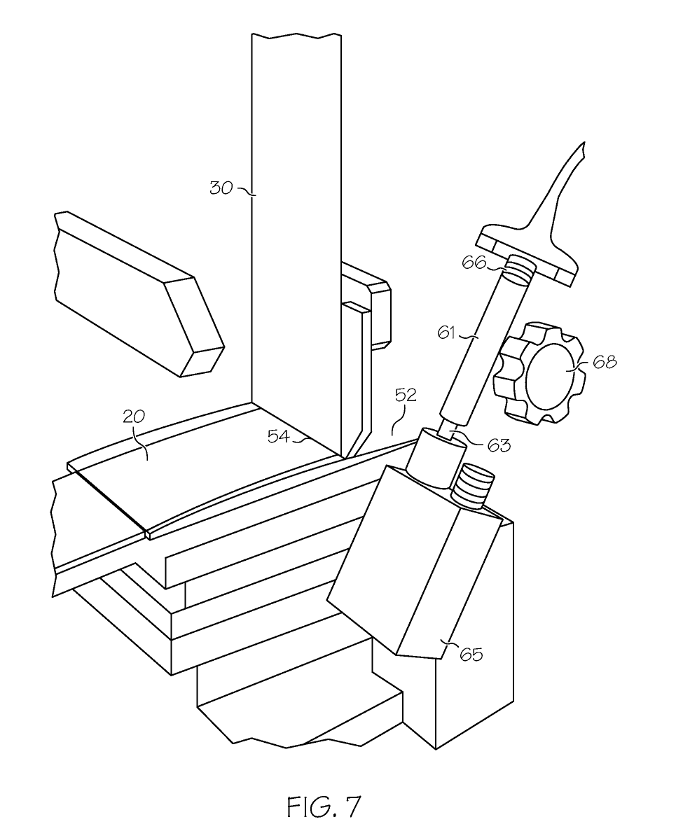

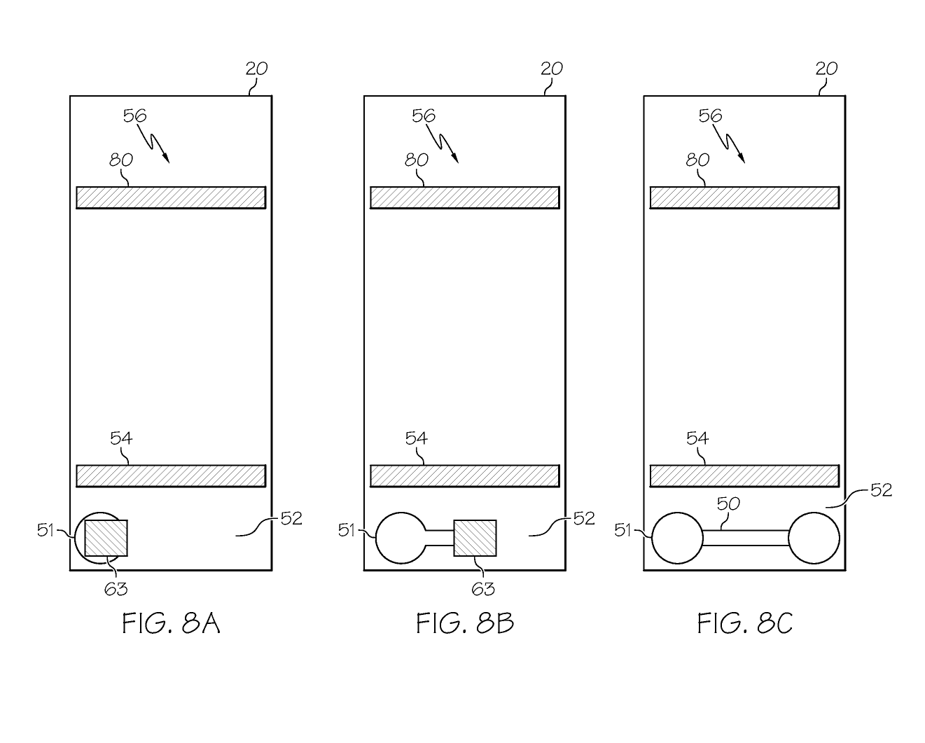

As is illustrated in FIG. 6, the metering gap 54 is a restriction wherein an outside surface 31 of the metering web 30 is brought into close proximity with the outside surface 21 of the transfer web 20, such that when the initial quantity of coating solution 50 is passed through the gap 54 at least some amount of excess coating solution 50b will adhere to the outside surface 31 of the advancing metering web 30 and be transported away. The coating solution that remains on the transfer web 20 after passing through the gap 54 defines a coating patch 50a that will have a substantially uniform thickness across its relevant working area 57. The working area 57 is the area of the patch which will be subsequently brought into contact with a medical device.

The coating patch 50a, is then advanced along the transfer web pathway 25 to a coating application area 56 wherein a medical device 100 is positioned. The advancement of the transfer web pathway 25 brings the patch 50a into contact with the medical device 100, which rolls through the working area of the patch 50a, thereby providing the external surface of the medical device with a substantially uniform coating.

### Initial Coating Solution Deposition.

Determining the initial quantity of coating solution 50 to be deposited at staging area 52 involves several factors, such as for example: the type and size of the medical device being coated, the desired concentration of therapeutic agent and carrier (within the coating solution) that the medical device is to include, the total drug content on the device, the surface area of the medical device to be coated, the speeds at which the metering and transfer webs advance, and the metering gap. The actual manner with which the deposition of the coating solution onto the outer surface 21 of the transfer web 20 may also vary. For example, it is possible to simply pour a pre-measured amount of coating solution from a container onto the transfer web by hand. In some embodiments the system utilizes an automated syringe 61 which is preloaded with one or more doses of coating solution. In some embodiments other solution dispensing mechanisms can include a slot or extrusion die, a slot-fed curtain, knife/rod/blade coating, gravure coating, deformable roll coating, etc.

In at least one embodiment, as depicted in FIGS. 7, the applicator tip 63 of a syringe 61 is initially positioned within a sealed chamber 65 to prevent evaporation of solvents contained in a pre-loaded syringe 61. The reservoir and/or syringe can be configured to warm or cool the coating solution prior to its use. Once the syringe 61 is loaded, a linear actuator, robotic arm or similar mechanism 68, repositions the applicator tip 63 over the staging area 52 of the transfer web 20. While staging area 52 is shown in FIG. 7 its function in receiving the initial quantity of coating solution is best show in FIGS. 8a-8c.

When the applicator tip 63 is positioned in the manner shown, an externally delivered positive pressure is applied to the plunger 66 and the initial quantity of coating solution 50 is deposited onto the transfer web 20. In some embodiments the coating solution is fed into the syringe 61 from a reservoir, and the syringe 61 (and/or applicator tip 63) is provided with an actuatable valve (or valves), which are opened to release a desired amount of coating solution 50.

In some embodiments, after each application of coating solution at least the portion of the syringe 61 including the tip 63 is cleaned; either manually or by positioning the syringe in a reservoir of cleaning solution (not shown).

The coating solution 50 is deposited in the form of an elongate bead 51 having a longitudinal axis 53 which upon deposition is substantially parallel to the width of the web 20 and/or substantially perpendicular to its length.

In some embodiments, an example of which is shown in FIG. 8c, the bead 51 is provided with end regions 55 having a comparatively greater volume of coating solution than the medial region 57. These end regions 55 will prevent the initial quantity of coating solution 50 from narrowing prematurely when passing through the metering gap 54.

As discussed above, in some embodiments the bead 51 is placed onto the outer surface 21 of the transfer web 20 while the transfer web is stopped (not advancing along the path way 25). In some embodiments the syringe 61, or more precisely the arm 68 is capable of applying the bead 51 onto the transfer web 20 during advancement of the web 20. Servomotors in the arm 68 are configured to move the applicator tip 63 across the width of the transfer web 20 as well as parallel to the advancing web 20 in order to compensate for the movement of the web 20 during the coating deposition.

Once the initial quantity of coating solution 50 is properly deposited onto the transfer web 20, the web is advanced in a “downstream” direction along the pathway 25 in order to pass the coating 50 through the metering gap 54.

Given the particularly volatile nature of the solvents typically employed in the formation of the coating solution, and in light of the tendency of such substances to evaporate, the staging area 52 is positioned fairly close to the gap 54 in order to minimize the exposure time of the coating.

In some embodiments however, this concern is mitigated by positioning the entire system in a temperature and vapor concentration controlled environment defined by a sealed housing or chamber. In some embodiments, such a chamber is provided only around and along the transfer pathway 25 and/or in regions of the pathway 25 wherein the coating solution is exposed. In at least one embodiment the one or more cover plates, are positioned adjacent to the transfer web 20. These cover plates extend across the width of the web 20 and along its length in desired regions, such as the staging area 52, the metering gap 54 and the coating application area 56. An inert gas or a solvent rich environment can be introduced along the pathway, such as within the aforementioned closed environment of a chamber, and/or within the coverage area of one or more cover plates.

### Metering Gap: Webs, Blade and Base.

As shown in FIG. 6, an aspect of the system 10 discussed herein is the use of a metering gap 54 to remove excess coating 50b from the initial quantity of coating solution 50 and provide a coating patch 50a of uniform thickness.

As mentioned above, the metering gap 54 is a region of the system where the transfer web pathway 25 and metering web pathway 35 can be made to intersect. While the height of the gap defined by the webs 20 and 30 can be adjusted to zero (e.g.

the webs 20 and 30 are in physical contact) the height is more commonly adjusted between about 1 μm to about 100 μm, or any desired height necessary to provide a coating patch 50a of proportional thickness. In at least one embodiment the metering gap 54 is configured to provide coating patch 50a with a thickness of about 5 μm to about 25 μm.

The thickness of the coating patch 50a resulting in the passage of a properly calibrated gap 54 will be approximately half the height of the gap, or in other words: the height of the gap is equal to about two times the coating thickness when both metering and transfer webs have the same linear velocity. Calibration of the gap by establishing uniformity of the zero position can be established optically by illuminating the gap from the downstream side toward the upstream side. The base is raised until light across the width of the web can no longer detected optically and the gap is manually adjusted in the cross-web direction by manipulating the blade fixture.

In some coating applications, the height of the gap 54 is between about 5 μm and 60 μm, depending on the thickness of the coating patch 50a desired to be applied to the medical device in accordance with the relationship mentioned above. In some embodiments the height of the gap can be adjusted by increments as slim as 0.01 μm.

As shown in FIG. 6, while the gap 54 itself is defined by the outer surfaces 21 and 31 of the respective transfer web 20 and metering web 30, the maintenance of the gap height is provided in-part by the presence of a metering blade 60 positioned against the inside surface 33 of the metering web. As shown in FIG. 1-4, blade 60 is mounted above the transfer web 20 at a downstream position from the staging area 52. In some embodiments the blade 60 includes alignment and/or repositioning mechanisms to allow the blade to be repositioned toward or away from the transfer web 20 as desired, in order to further restrict or open the gap 54. The blade 60 can be positioned at any angle relative to the transfer web 20. Adjustment of the blade 60 in this manner results in a corresponding adjustment of the metering web 30, as the web 30 is held under tension against the blade 60. The tension of the metering web 30 against the blade 60 is automatically adjusted to be maintained during the gap height adjustment.

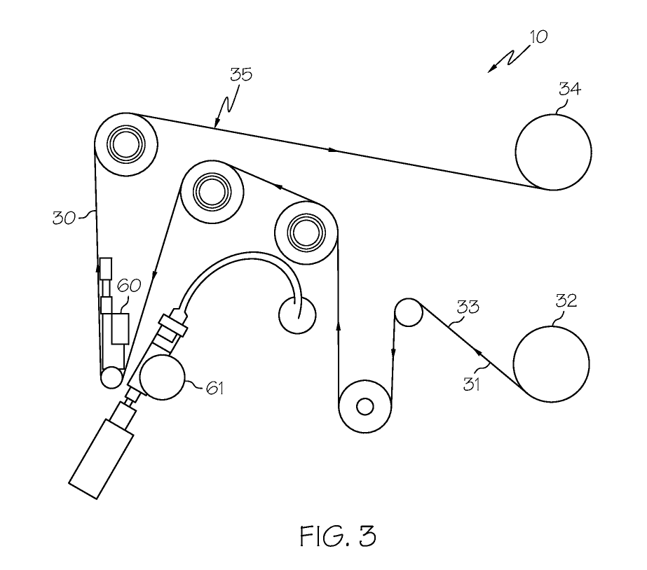

Blade 60 is provided with an engagement surface or edge 62. At least a portion of the edge 62 is biased against the inside surface 33 of metering web 30 along an edge line 64. In the manner previously described and shown in FIG. 3 and FIG. 6, the metering web 30 moves around the edge 62 as the web 30 advances along the metering web pathway 35. The outside surface 31 of the metering web that is opposite the edge line 64 defines the upper portion of the gap 54, and is positioned to contact the initial quantity of coating solution 50, and draw off the excess coating solution 50b in the manner described above.

In some embodiments, the quantity of excess coating 50b that is removed via the gap 54 can be controlled by moving the metering web 30 at a greater speed than the transfer web to through the gap 54. By independently modifying the speed of the webs 20 and 30, a greater degree of control over the coating thickness is provided.

As depicted in FIG. 6, the lower portion of the gap 54 is defined by the outer surface 23 of the transfer web 20. In some embodiments the transfer web 20 is provided with sufficient tension and/or material strength to provide, by itself, the lower definition of the gap 54. In some embodiments however, a base or substrate 70 is positioned under the transfer web 20 to assist the web 20 in establishing and maintaining the height of gap 54.

In at least the region of the gap 54 and/or the staging area the base 70 is a member or surface of a member constructed of any material desired that is sufficient to provide a rigid backing against the inner surface 23 of the transfer web 20, so that the uniformity of the outer surface 31 of the transfer web 30 is not compromised during the passage of the coating solution (in the case of the gap 54) or deposition of the coating solution (in the case of the staging area 52) thereon.

In some embodiments, at least the portion of the base 70 underlying the gap 54 and/or staging area 52 is constructed of a non-compliant material such as stainless steel, ceramic, tool steel, tungsten carbide, steel alloys, diamond like carbon, etc. In some embodiments the blade 60, or at least the edge 62 of the blade, is constructed of the same or similarly rigid or hard materials as the base 70.

In some embodiments, the base 70 or at least a portion of the base 70 corresponding to the coating application area 56 can be comprised of hard materials (such as for example those materials previously mentioned), or of somewhat compliant materials having a Durometer (Shore A) hardness value of 10 to 90. Some examples materials suitable for use in the composition of a compliant base region include, but are not limited to PTFE, polycarbonate, neoprene, polyurethane, etc.

In some embodiments, the position of the base 70 relative to the position of the blade 60 is adjustable. By moving the base 70 toward or away from the blade 60 the gap height can be adjusted. The tension of the transfer web 20 against the base 70 is automatically adjusted to be maintained during the gap height adjustment. In some embodiments both the blade 60 and base 70 are independently adjustable.

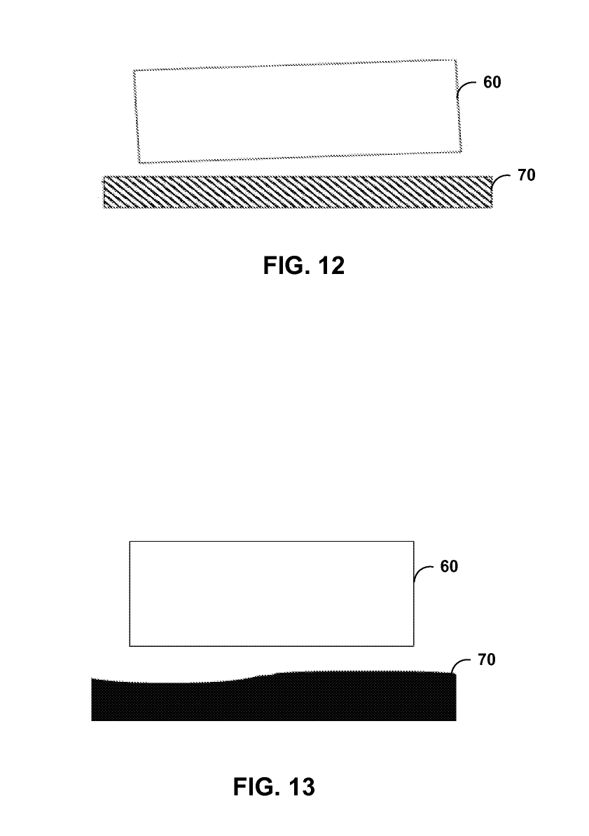

It is of significant importance that the blade 60 and base 70 be free of surface or alignment defects, which could negatively impact the thickness uniformity of the coating patch. For example, FIG. 12 depicts a longitudinal cross-section of the metering gap (e.g. across the width of the transfer wed) wherein the blade 60 is misaligned relative to the base 70; it should be readily understood that the formation of a coating patch utilizing such a gap would not have a uniform thickness.

Similarly, should one or both of the blade 60 and base 70 include significant surface imperfections, such imperfections will lead to unacceptably irregular patch thickness. An example of such a surface imperfection in base 70 is depicted in FIG. 13.

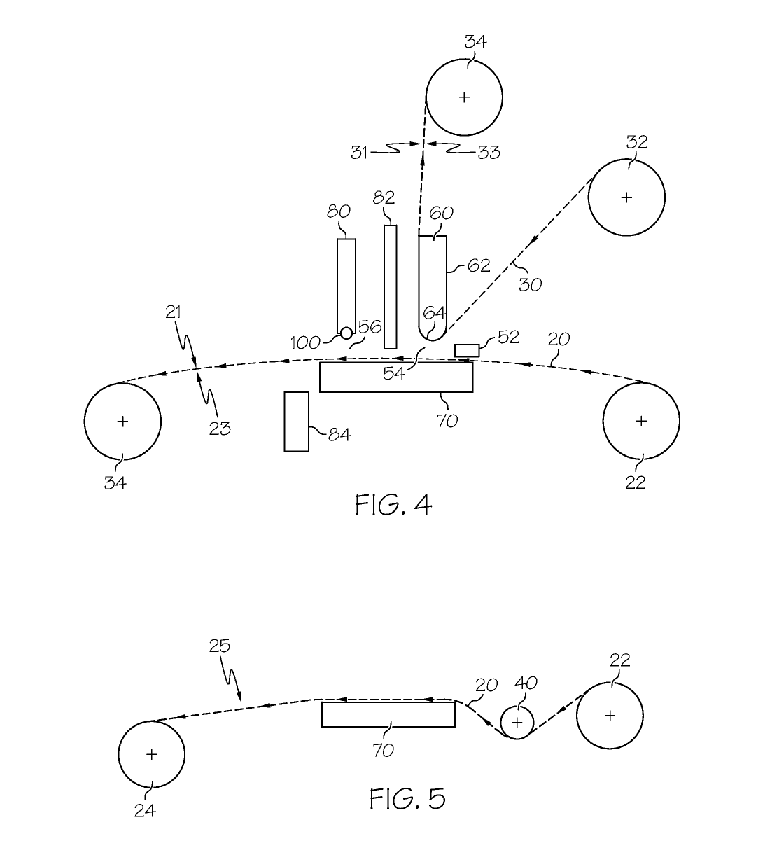

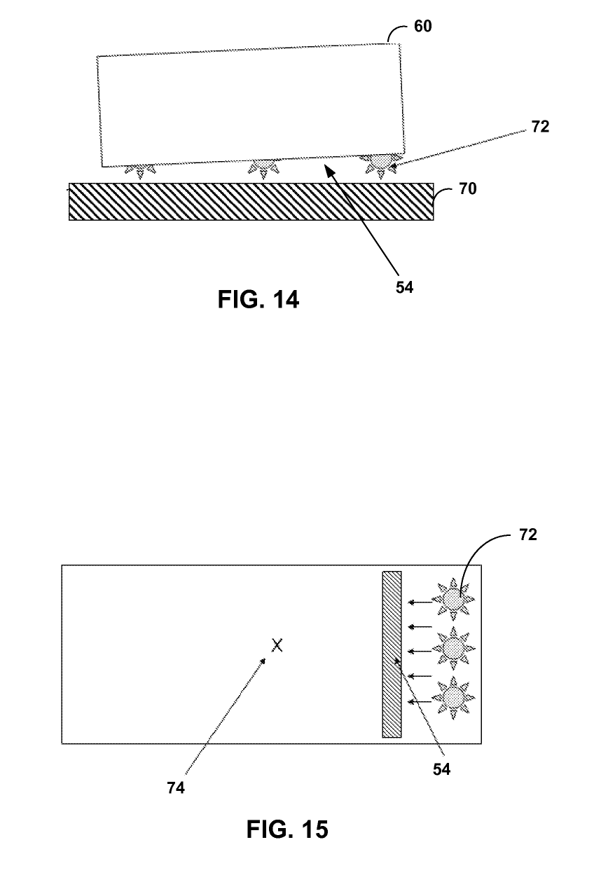

Such alignment and/or surface imperfections can be readily detected. In at least one embodiment, light from light source 72 is passed through the gap 54, such as depicted in FIG. 14, and inconsistencies in the quantity/intensity of light passed through the gap along its width, are observed. As seen in FIG. 15, lighting from a backside of a blade using light source 72 allows an operator looked at position 74 to visually assess the uniformity of the gap 54 when the gap 54 is set to what is believed to be zero. If not aligned, light is observed at one end of the blade but not the other. The detection of such inconsistencies establishes to the user or an automatic detection system 82 (as depicted in FIG. 4) that the gap is not properly configured. Such a determination can be part of a feedback loop which triggers an alarm, and/or shut down of the system 10.

Because the base 70 and blade 60 will likely come into direct contact with the relevant webs during advancement of the web along their pathways 25 and 35, in at least some embodiment, the base 70 and/or the blade 60 (or at least the portion(s) of the base and/or blade which directly contacts the web) is provided with a coating of one or more materials having a particularly low coefficient of friction. Some examples of such coating materials include but are not limited to: poly(dimethyl siloxane) (PDMS), PTFE, etc. The utilization of such surface treatments allow ease of transport of the web and minimize risk of web wrinkling in the gap 54.

In some embodiments a lubricant (via a lubricant applicator and reservoir) may also be provided on the surface of the base 70 adjacent to the inner surface 23 of the transfer web 20.

While the material characteristics and proper alignment of the blade 60 and base 70 in defining a uniform gap opening can certainly affect the formation and consistency of the coating patch thickness. It must also be noted that the surface smoothness of the web surfaces 21 and 31 is also a significant factor in ensuring the uniformity of the coating patch.

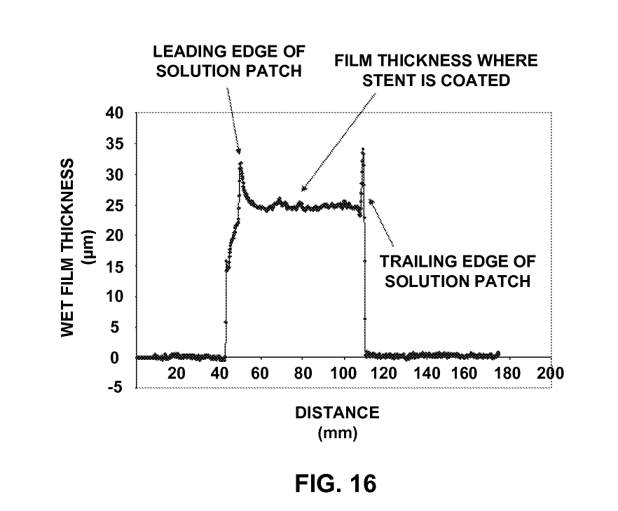

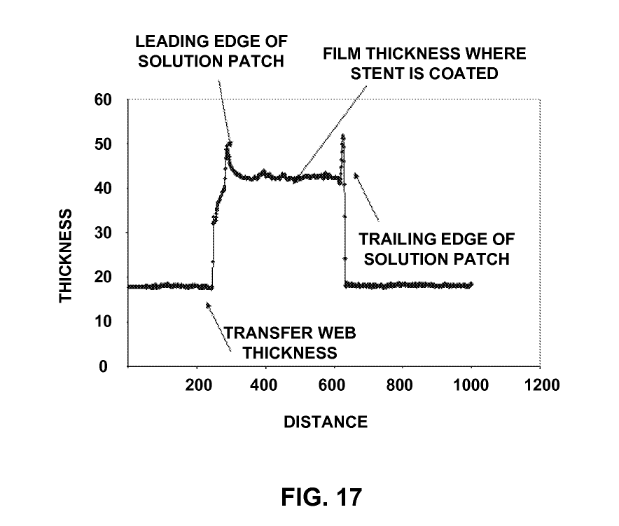

FIGS. 16 and 17 depict, for at least one embodiment, the thickness uniformity of the coating patch 50a along its length, as well as the relationship of the patch thickness to the thickness uniformity of the outer surface 21.

### Coating Patch

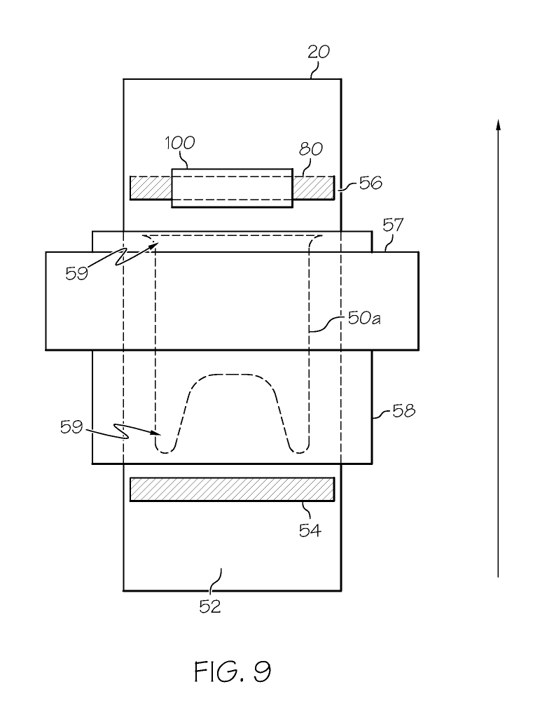

The coating patch 50a which exits the metering gap 54 in a downstream direction will have a significantly greater area than the bead 51 which was initially deposited at the staging area 52 upstream of the gap 54. The coating patch 50a may have a variety of shapes and configurations depending on the size and shape of the original bead 51 as well as other factors. For any coating patch made in accordance with the present invention, some portion of the patch will define a useable or working portion 57, there may also be present a portion of extraneous material 59. For example, the coating patch 50a shown in FIG. 9 has a shape which reflects the barbell-like shape of the original bead 51 (see FIG. 8c). In this embodiment, the working portion 57 is located behind the downstream edge 58 of the patch 50a and extending approximately 50% of the patch's length.

The distinction of the working portion 57 and the extraneous portion 59 is based on the thickness of the coating patch 50a. In the area of the working portion 57 the thickness of the coating will be substantially uniform, whereas in the area of the extraneous portion 59 the thickness may vary to an unacceptable degree. For example if the metering gap 54 is set to provide a coating thickness of 20 μm (see discussion above regarding gap height), then the working portion 57 of the coating patch 50, such as is shown in FIG. 9 will have a thickness of 20 μm+/−0.25 μm. The thickness of the extraneous material 59 may have areas outside of this range.

The working portion 57 of the coating patch 50a is also distinct from the extraneous portion 59 in that it is only the working portion 57 of the coating that a medical device 100 is brought into contact with.

### Coating Application Area

Continuing its downstream journey along the transfer web pathway 25, following the passage through the metering gap 54, the coating patch 50a is brought to the coating application area 56 of the current system 10. In at least one embodiment the coating application area 56 comprises a medical device retaining mechanism 80 which extends substantially parallel to the width of the transfer web 20. In some embodiments mechanism 80 includes a mandrel, pin, spring and/or other device suitable for mounting a medical device 100 such as a stent, balloon, catheter or catheter component, etc. thereon.

In some embodiments the mechanism 80 is moveable in a lateral direction toward and away from the transfer web 20. When in a non-engaged (away) position the mechanism does not contact the web 20 or the coating 50a. When in this position, a medical device 100 may be loaded onto or removed from the mechanism 80. When in the engaged (toward the web) position a medical device 100 mounted on the mechanism is in contact with the web 20 and/or the working portion 57 of the coating patch 50a.

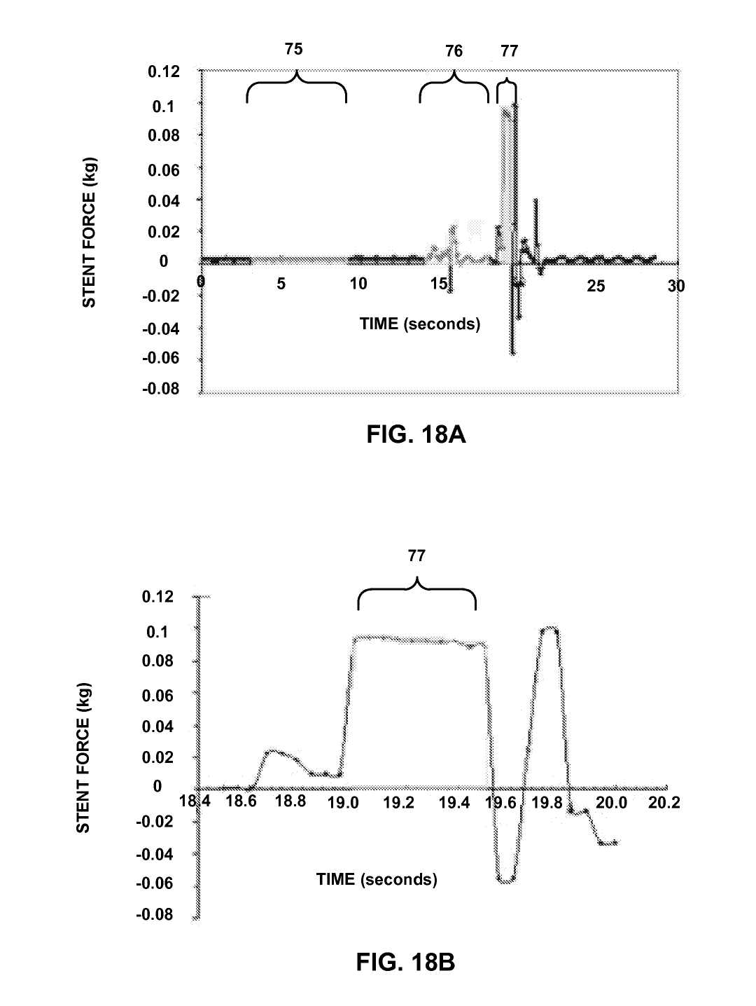

As can be seen in FIGS. 18A and 18B, the force applied to a medical device 100, in this specific example a stent, varies during the coating process in the manner shown. FIGS. 18A and 18B depict the stent force, measured by a load cell, versus time. Time sequence 75 is when the solution is being dispensed. Time sequence 76 is when the solution is being metered. Time sequence 77 is when the stent is in contact with the transfer web.

In some embodiments mechanism 80 includes a load cell which provides a force feed back loop to a controller (not shown). The load cell measures the force the medical device mounted on the mechanism 80 applies to the web 20 during the coating application. When a predetermined force is reached, the mechanism 80, via the load cell, keeps the medical device in the engaged position for a set amount of time (e.g. time sufficient to complete one or more full rotations of the device 100 through the working portion 57 of the coating patch 50a).

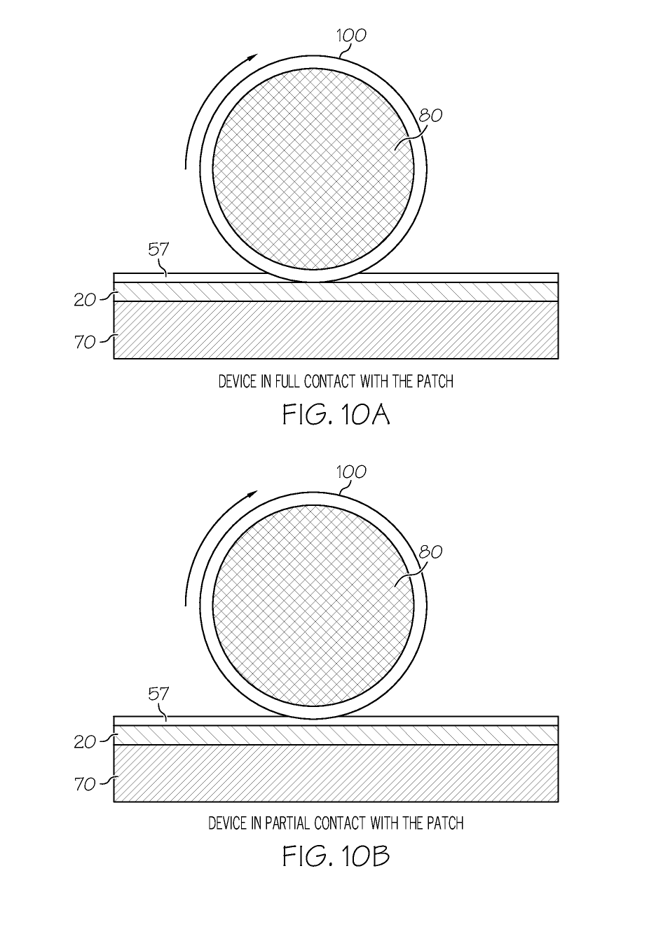

When in the engaged position, the advancement of the transfer web 20 will impart a corresponding rate of rotation in the mechanism 80 and thus the medical device 100 mounted thereon. As is depicted in FIG. 1b and FIG. 1a, the medical device 100 will be held in position over, and at least partially within, the coating patch 50a during this rotation. FIG. 10b, depicts an embodiment of the invention wherein the medical device 100 is held in position to roll through only a portion of the thickness of the coating patch 50a. FIG. 10a, depicts an embodiment of the invention wherein the medical device 100 is rolled through the entire thickness of the coating patch 50a.

Upon completion of one or more rotations of the medical device 100 through the coating patch 50a, the mechanism 80 and thus the medical device 100 is move to the unengaged position. The rotation of the medical device 100 through the working portion 57 of the coating patch 50a, will result in the medical device receiving a substantially uniform thickness of coating along the surface of the device 100 that has rolled through the coating 50a.

In some embodiments the medical device 100 is retained in the unengaged position for a period of time sufficient to allow the coating applied thereto to properly dry or cure. In some embodiments a heat source can be directed toward the medical device to encourage the drying/curing process. In some embodiments the dried/cured coating on the medical device will have a thickness of about 1.5 μm to about 14 μm.

Once the coating is properly cured the mechanism 80 can reengage the medical device into a second coating patch in order to allow the medical device 100 to have multiple coating layers applied thereto. By repeating the coating process the medical device 100 can thus be provided with any number of similar or different coating layers as desired. Layers of coating solution can include no therapeutic agent or, one or more therapeutic agents.

In some embodiments, mechanism 80 includes a drive mechanism which can provide the mechanism with rotation in either direction, independent of the direction and speed of the transfer web 20.

In some embodiments the working portion 57 of the coating patch 50a, has a length sufficient to allow the medical device 100 to complete a single circumferential rotation therethrough. In some embodiments the working portion 57 of the coating patch 50a has a length sufficient to allow the medical device 100 to complete multiple circumferential rotations therethrough in order to accumulate a thicker coating onto the device's surface.

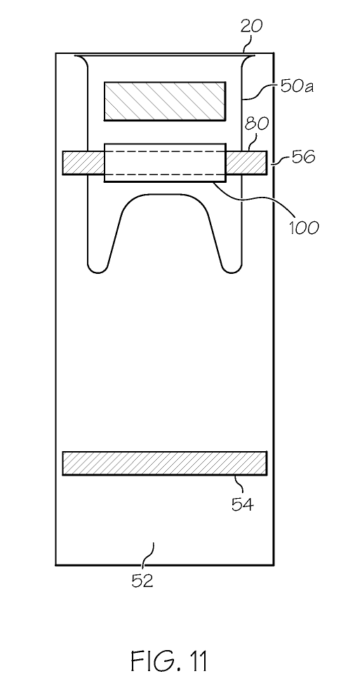

After the medical device 100 has completed its interaction with the coating patch 50a, any topographical pattern present on the surface of the medical device (for example, the pattern of stent members and cell openings in a tubular stent) will be reflected in the post-application patch, such as in the manner shown in FIG. 11.

In at least one embodiment the imprinted coating patch can by analyzed using differential interference contrast imaging, dark field illumination or other techniques. Enhanced contrast of the image will depict the topography of the medical device imprinted within the coating. By analyzing this depiction a user can determine if the coating applied to the medical device is uniform. A visualization system 84 suitable for providing such imagery analysis is shown in FIG. 4 wherein it is positioned adjacent (downstream) of the coating application area 56.

In some embodiments the coated medical device itself, or more specifically the coating applied thereto, can be analyzed using spectral reflectance, low coherence interferometry, white light interferometry, confocal aberration or similar techniques to determine the thickness of the coating at various locations on the device to determine the concentration and/or distribution of the drug content contained within the coating.

### Coatings and Therapeutic Agents

As mentioned extensively above, the present invention is of particular use in applying coatings to a medical device. While the coating solution contains a variety of substances such as the solvents used to dissolve the therapeutic agent, and often one or more polymer agents as well. Of particular concern to the physician and patient however, is the therapeutic aspect of the coating. Embodiments of the present invention can utilize any therapeutic agent in the formation of the coating solution described above. The term “therapeutic agent” as used herein encompasses drugs, genetic materials, and biological materials and can be used interchangeably with “biologically active material”. The term “genetic material” means DNA or RNA, including, without limitation, DNA/RNA encoding a useful protein stated below, intended to be inserted into a human body including viral vectors and non-viral vectors.

The term “biological materials” include cells, yeasts, bacterial, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferring, cytotactin, cell binding domains (e.g., RGD), and tenascin. Examplary BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, urokinase, and PPack (dextrophenylalanine praline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucorticoids, betemethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antoagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-convertin enzyme (ACE) inhibitors including captropril and enalopril, statins and related compounds; and
macrolides such as sirolimus or everolimus;

Other therapeutic agents include nitroglycerin, nitrous oxides, nitric oxides, antibiotics, aspirins, digitalis, estrogen, estradiol and glycosides. Exemplary therapeutic agents include anti-proliferative drugs such as steroids, vitamins, and restenosis-inhibiting agents. Exemplary restonosis-inhibiting agents include microtubule stabilizing agents such as Taxol®, paclitaxel (i.e., paclitaxel, paxlitaxel analogs, or paclitaxel derivatives, and mixtures thereof). For example, derivatives suitable for use in the medical devices include 2′-succinyl-taxol, 2′-succinyl-taxol triethanolamine, 2′-glutaryl-taxol, 2′glutaryl-taxoltriethanolamine salt, 2′-O-ester with N-(dimethylaminoethyl) glutamine, and 2′-O-ester with N-(dimethylaminoethyl) glutamide hydrochloride salt.

Other exemplary therapeutic agents include tacrolimus; halafuginone; inhibitors of HSP90 heart shock proteins such as geldanamysin; microtubule stabilizing agents such as epothilone D; phosphodiesterase inhibitors such as cliostazole; Barkct inhibitors; phospholamban inhibitors; and Serca 2 gene/proteins. In yet another embodiment, the therapeutic agent is an antibiotic such as erythromycin, amphotericin, rapamycin, adriamycin, etc.

In some embodiments, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In some embodiments, the therapeutic agents for use in the medical devices can be synthesized by methods well known to one skilled in the art. Alternatively, the therapeutic agents can be purchased from chemical and pharmaceutical companies.

Where the therapeutic agent includes a polymer agent, the polymer agent may be a polystyrene-polyisobutylene-polystyrene triblock copolymer (SIBS), polylactic acid (PLA) poly-lactic-glycolic acid, poly(D,L-lactide-glycolide), polyethylene oxide, silicone rubber; and/or other biodegradable and biostable polymers such as for example: poly(n-butyl methacrylate) (PBMA), poly(vinylidene fluoride-hexafluoropropylene) (PVDF-HFP); and/or any other suitable substrate.

In some embodiments, a polymer agent such as one the aforementioned examples, is a first or initial coating solution, which is initially applied to the medical device 100 in the manner described above (e.g. the coating solution 50 is applied to the transfer web 20, advanced, formed into patch 50a, through which the medical device 100 is rolled through, etc.).

In embodiments where the therapeutic agent and/or the polymer are dissolved in a solution of solvent(s), the solvent or solvents can be selected from at least one member of the group consisting of: Acetone, methyl ethyl ketone (MEK), methyl iso-butyl ketone (MIBK), tetrahydrofuran (THF), butyl acetate, ethyl acetate, dimethyl formamide (DMF), dimethyl sulfoxide (DMSO), cyclohexanone, water, and ethanol.

### Stent Pre-Treatment

As should be apparent from the above description that the coating process described herein is capable of applying a wide range of coating weights. In some embodiments however, such as those involving very low coating weights the weight of the coating along the surface of the stent may vary more than desired and voids in the coating may occur. To address these concerns, in some embodiments the medical device 100 is subjected to a plasma treatment prior to contacting the coating patch 50a.

In some embodiments the medical device is plasma treated and then coated using system 10 within 24 hours of the plasma treatment.

In order to minimize handling of the medical device 100, in at least one embodiment (an example of which is depicted in FIG. 4) the medical device 100 is retained on a retaining mechanism 80 within a plasma chamber 83 for plasma treatment. The retaining mechanism 80 is moveable from a position within the plasma chamber 83 to a position adjacent to the coating application area 56. Following the plasma treatment the retaining mechanism 80 and medical device 100 is repositioned to the coating application area 56.

In at least one embodiment the stent is subjected to a vacuum plasma technique such as provided by the March plasma system. In a vacuum plasma system, the medical device 100 is positioned in a chamber at low pressure and multiple parts or the entire stent is processed simultaneously.

In at least one embodiment an atmospheric technique such as a Brush plasma system is utilized, wherein a stream of gas plasma is passed over a surface or surface of the stent to treat it.

The main difference between the two treatment options mentioned above is that the vacuum process mostly treats the surface by use of ions, where as, the atmospheric process treats by use of radicals.

In some embodiments the plasma treatment, to which the medical device 100 is subjected, utilizes a combination of gasses such as for example hydrogen and oxygen. In some embodiments an inert gas such as for example: argon and/or nitrogen are utilized.

It is noted that the aforementioned plasma treatment processes can be used to treat polymer substrates in addition to metals.

In the present invention a plasma treating step has been shown to reduce the number of coating voids compared to web coating an untreated medical device such as a stent. This effect is greater at lower coating weights where the thickness of the solution patch that the stent is rolled through is thinner.

In one example wherein the medical device 100 was a stent, the stent was constructed of Platinum Chrome Alloy (a platinum rich stainless steel) and was subjected to atmospheric plasma treatment, the amount of oxide and hydroxyl groups measured on the surface were increased. An increase in —COOH groups on the surface was also observed which is believed to increase adhesion of the coating to the stent. The oxide, hydroxy and carboxyl groups are especially advantageous in the adhesion of biodegradable coatings containing carboxyl or hydroxyl end groups.

This completes the description of the preferred and alternate embodiments of the invention. Those skilled in the art may recognize other equivalents to the specific embodiment described herein which equivalents are intended to be encompassed by the claims attached hereto.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

1. A system for applying a coating solution to a medical device, the system comprising:
a transfer web, the transfer web defining an inside surface and an outside surface, the transfer web extending along a transfer web pathway, the transfer web being moveable in an upstream and downstream direction along the transfer web pathway;
a metering web, the metering web defining an inside surface and an outside surface, the metering web extending along a metering web pathway;
a coating deposition mechanism, the coating deposition mechanism constructed and arranged to deposit a quantity of coating solution onto a staging area, the staging area comprising a region of the outside surface of the transfer web,
a metering gap, the metering gap being defined by the outside surface of the transfer web and the outside surface of the metering web, the metering gap being positioned downstream from the staging area; and
a medical device support, the medical device support positioned at a coating application region being defined by the outside surface of the transfer web downstream from the metering gap.

2. The system of claim 1 wherein the metering web is moveable in an upstream and downstream direction along the metering web pathway.

3. The system of claim 1 further comprising a blade, the blade comprising an edge, the metering web pathway extending around at least a portion the edge, the inside surface of the metering web contacting the at least a portion of the edge along an edge line.

4. The system of claim 1 wherein the coating deposition mechanism comprises a syringe, the syringe being in fluid communication with a coating solution reservoir, the syringe having a tip, the tip being positioned adjacent to the transfer web at the staging area to deposit an initial quantity of coating solution from the coating solution reservoir thereon.

5. The system of claim 1 wherein the initial quantity of coating solution applied to the transfer web defines a barbell-like shape.

6. The system of claim 1 wherein the initial quantity of coating solution is advanced through the metering gap to form a coating patch, the coating patch having a working area, the working area being defined by an area of coating solution having uniform thickness.

7. The system of claim 6 wherein the metering gap has a height of about 1 μm to about 100 μm.

8. The system of claim 7 wherein the uniform thickness of the coating patch is about half the height of the metering gap.

9. The system of claim 7 wherein the uniform thickness of the coating patch is about about 5 μm to about 25 μm.

10. The system of claim 1 wherein the medical device support includes a medical device rotatably disposed thereon, the medical device being positioned immediately adjacent to the transfer web.

11. The system of claim 10 wherein the medical device is selected from the group consisting of: a stent, a balloon, a catheter, and any combinations thereof.

12. An apparatus for coating a medical device, the apparatus comprising:
a transfer web, the transfer web extending from a transfer web source roller to a transfer web receiving roller;
a metering web, the metering web extending from a metering web source roller to a metering web receiving roller;
at least one mechanism for advancing the transfer web in a downstream direction from the transfer web source roller to the transfer web receiving roller and advancing the metering web from the metering web source roller to the metering web receiving roller;
a metering gap, a region of the transfer web and a region of the metering web defining the metering gap;
a coating solution applicator, the coating solution applicator positioned adjacent to a staging area of the transfer web at a position upstream of the gap,
a medical device retaining mechanism, the medical device retaining mechanism positioned adjacent to a coating application area of the transfer web, at a position downstream from the gap.

13. A method for applying a coating to a medical device comprising:
providing a coating apparatus, the coating apparatus having:
a transfer web, the transfer web extending from a transfer web source roller to a transfer web receiving roller;
a metering web, the metering web extending from a metering web source roller to a metering web receiving roller;
a gap, a region of the transfer web and a region of the metering web defining the gap;
a coating solution applicator, the coating solution applicator positioned adjacent to a staging area of the transfer web at a position up stream of the gap,
a medical device retaining mechanism, the medical device retaining mechanism positioned adjacent to a coating application area of the transfer web, at a position downstream from the gap;
depositing at the staging area an initial quantity of coating solution from the coating solution application onto the transfer web;
advancing the transfer web along a transfer web pathway in a downstream direction thereby transporting the initial quantity of coating solution from the staging area to the gap;
advancing the metering web along a metering web pathway;
advancing the initial quantity of coating solution through the gap, wherein the metering web contacts the initial quantity of coating solution, the metering web drawing off an excess amount of coating solution from the initial quantity of coating solution resulting in the formation of a coating patch on the transfer web;
advancing the coating patch to the medical device retaining mechanism, the medical device retaining mechanism including a medical device, the medical device being freely rotatable relative to the transfer web; and
rolling the medical device through a working portion of the coating patch as the coating patch passes by the medical device retaining mechanism by advancement of the transfer web.
